(19) United States
(12) Patent Application Publication
(10) Pub. No.: US 2008/0221497 A1
(43) Pub. Date: Sep. 11, 2008

(54) Use of Arginine and Like Substances and methylglyoxal and Like Substances in Dialysis Machines

(76) Inventor: George M. Haik Jr., New Orleans, LA (US)

Correspondence Address: GARVEY SMITH NEHRBASS & NORTH, LLC, LAKEWAY 3, SUITE 3290, 3838 NORTH CAUSEWAY BLVD., METAIRIE, LA, 70002, US

(21) Appl. No.: 11/568,378

(22) PCT Filed: Apr. 26, 2004
(86) PCT No.: PCT/US04/12945

§ 371 Date: Nov. 5, 2007

(51) Int. Cl. A61M 1/34; C07C 229/02

(52) U.S. Cl. 604/6.09; 562/433

(57) ABSTRACT

A kidney dialysis filter based on arginine attached to a cellulose polymer or a chitin polymer or a dendrimer polymer (or other nonsoluble carrier polymer—large enough that they will not go from dialysis machine to patient by going through the filtration membrane or by dissolving and entering the blood stream of the patient—or a non-biological oligomer), a kidney dialysis machine using the filter, and a method of producing the filter by, for example, attaching an arginyl grouping or moiety to a cellulose polymer or a chitin polymer or a dendrimer polymer (or other nonsoluble carrier polymer or a non-biological oligomer as mentioned above), can all be used to help prevent biological damage which might be caused by toxic carbonyl containing compounds and/or dicarbonyl containing compounds.

### CROSS-REFERENCE TO RELATED APPLICATIONS

In the US, this is a continuation-in-part patent application of my co-pending U.S. patent application Ser. No. 09/435,270, filed 5 Nov. 1999, which is a continuation-in-part patent application of my co-pending U.S. patent application Ser. No. 09/079,329, filed May 14, 1998 and of my U.S. patent application Ser. No. 08/848,414, filed May 7, 1997, which is a continuation-in-part patent application of U.S. patent application Ser. No. 08/745,060, filed Nov. 7, 1996, and of International Patent Application No. PCT/US96/17821, filed Nov. 7, 1996, published as WO 97/17081 on 15 May 1997, all incorporated herein by reference.

In the US, this is also a continuation-in-part patent application of my co-pending U.S. patent application Ser. No. 09/079,329, filed May 14, 1998, which is a continuation-in-part patent application of my U.S. patent application Ser. No. 08/848,414, filed 7 May 1997.

My International Patent Application No. PCT/US98/10057, filed 14 May 1998, published as WO 98/51260 on 19 Nov. 1998 is also incorporated herein by reference.

My U.S. Provisional Patent Application Ser. No. 60/006,304, filed 7 Nov. 1995, is hereby incorporated by reference.

My U.S. Provisional Patent Application Ser. No. 60/046,430, filed 14 May 1997, is hereby incorporated by reference.

My U.S. Provisional Patent Application Ser. No. 60/059,420, filed 31 Jul. 1997, is hereby incorporated by reference.

My U.S. Provisional Patent Application Ser. No. 60/107,469, filed 6 Nov. 1998, is hereby incorporated by reference.

In the US, priority of each of these patent applications is hereby claimed.

### STATEMENT REGARDING FEDERALLY SPONSORED RESEARCH OR DEVELOPMENT

Not applicable

### REFERENCE TO A “MICROFICHE APPENDIX”

Not applicable

### BACKGROUND OF THE INVENTION

1. Field of the Invention

The present invention relates to dialysis machines. More particularly, the present invention relates to filters for dialysis machines.

2. General Background of the Invention

Dialysis machines are used to filter harmful components out of a patient's blood by cycling the blood through a filter or filters in the machines. See my International Patent Publication No. WO 98/51260 for information about the use of arginine and like substances to treat persons by administering arginine to the persons or by treating blood and/or blood products in a dialysis system for return to the subject comprising administering to a subject suffering from a condition associated with toxic carbonyl containing compounds and/or dicarbonyl containing compounds a therapeutically effective dose to a subject's blood and/or blood products of a blocking agent selected from the group consisting of arginine, substituted arginine, or modified arginine.

### BRIEF SUMMARY OF THE INVENTION

In Science Vol. 277, 19 Sep. 1997, Dr. James C. Nelson et al. revealed in an article entitled “Solvophobically Driven Folding of Nonbiological Oligomers” a phenylacetylene oligomer on page 1794 FIG. 1(A) which the present inventor believes could be used and/or modified for use as a carrier molecule for 1) methylglyoxal, glyoxal, malondialdehyde, or similar molecules for use in hemodialysis of uremic patients to remove compounds with certain amino groupings such as arginine, lysine, cysteine or urea not to exclude other aminated compounds or 2) arginine or similar molecules for use in hemodialysis of diabetic patients to remove toxic dicarbonyl compounds such as methylglyoxal, glyoxal, deoxyglucasone and certain advanced glycation end-products.

Arginine could be attached to this molecule (the solvophobic helical macromolecule published by Dr. James Nelson and his colleagues at the University of Illinois in the journal Science) and the resulting compound used in a renal dialysis machine to remove toxic carbonyl and dicarbonyl compounds from the blood of diabetic patients on renal dialysis. Conversely, a dicarbonyl compound such as methylglyoxal could be attached to the same solvophobic helical backbone and employed in dialysis machines to remove arginine, lysine, cysteine and nitrogenous waste products from the blood of uremic, liver failure patients.

The present invention includes a method of treating diabetic dialysis patients with a phenylacetylene oligomer or other oligomer or macromolecule or dendrimer modified with an arginine side chain, cysteine side chain and/or lysine side chain for removal of toxic carbonyl and/or dicarbonyl compounds such as methylglyoxal, glyoxal, malondialdehyde, deoxyglucasone and advanced glycation end-products by placement of the modified oligomer, macromolecule or dendrimer in the filtration system of the dialysis machine.

The present invention also includes a method of treating uremic dialysis patients with a phenylacetylene oligomer or other oligomer or macromolecule or dendrimer modified with a side chain consisting of a carbonyl, and/or dicarbonyl such as methylglyoxal, glyoxal, malondialdehyde, deoxyglucasone or phenylglyoxal for removal of aminated compounds such as arginine, cysteine, lysine, creatinine or urea not to exclude other aminated compounds by placement of the modified oligomer, macromolecule or dendrimer in the filtration system of the dialysis machine.

The present invention includes a dialysis machine for use in treating diabetic dialysis patients, the dialysis machine having a filtration system, the filtration system having therein a phenylacetylene oligomer or other oligomer or macromolecule or dendrimer modified with an arginine side chain, cysteine side chain and/or lysine side chain for removal of toxic carbonyl and/or dicarbonyl compounds such as methylglyoxal, glyoxal, malondialdehyde, deoxyglucasone and advanced glycation end-products.

The present invention includes a dialysis machine for use in treating uremic dialysis patients, the dialysis machine having a filtration system, the filtration system having therein a phenylacetylene oligomer or other oligomer or macromolecule or dendrimer modified with a side chain consisting of a carbonyl, and/or dicarbonyl such as methylglyoxal, glyoxal, malondialdehyde, deoxyglucasone or phenylglyoxal for removal of aminated compounds such as arginine, cysteine, lysine, creatinine or urea not to exclude other aminated compounds.

The present invention also comprises a kidney dialysis filter based on arginine attached to a cellulose polymer or a chitin polymer or a dendrimer polymer (or other nonsoluble carrier polymer—large enough that they will not go from dialysis machine to patient by going through the filtration membrane or by dissolving and entering the blood stream of the patient—or a non-biological oligomer), a kidney dialysis machine using the filter, and a method of producing the filter by, for example, attaching an arginyl grouping or moiety to a cellulose polymer or a chitin polymer or a dendrimer polymer(or other nonsoluble carrier polymer—large enough that they will not go from dialysis machine to patient by going through the filtration membrane or by dissolving and entering the blood stream of the patient—or a non-biological oligomer). For example, it is possible to attach arginine via its carboxyl group to cellulose acetate polymer after the removal of the acetate grouping or moiety with the use of a coupling agent, such as 1-cyclohexyl-3-(2-morpholino-ethyl)carbodiimide metho-p-toluenesulfonate or N,N′-dicyclohexylcarbodiimide or similar carbodiimide compound not to exclude other coupling agents. Also, one could produce the filter by attaching active arginyl chloride to cellulose acetate polymer after the removal of the acetate grouping or moiety.

### DETAILED DESCRIPTION OF THE INVENTION

The present invention comprises a kidney dialysis filter based on arginine attached to a cellulose polymer or a chitin polymer or a dendrimer polymer (or other nonsoluble carrier polymer—large enough that they will not go from dialysis machine to patient by going through the filtration membrane or by dissolving and entering the blood stream of the patient—or a non-biological oligomer), a kidney dialysis machine using the filter, and a method of producing the filter by, for example, attaching an arginyl grouping or moiety to a cellulose polymer or a chitin polymer or a dendrimer polymer (or other nonsoluble carrier polymer—large enough that they will not go from dialysis machine to patient by going through the filtration membrane or by dissolving and entering the blood stream of the patient—or a non-biological oligomer). For example, it is possible to attach arginine via its carboxyl group to cellulose acetate polymer after the removal of the acetate grouping or moiety with the use of a coupling agent, such as 1-cyclohexyl-3-(2-morpholino-ethyl)carbodiimide metho-p-toluenesulfonate or N,N′-dicyclohexylcarbodiimide or similar carbodiimide compound not to exclude other coupling agents. Also, one could produce the filter by attaching active arginyl chloride to cellulose acetate polymer after the removal of the acetate grouping or moiety.

A possible way of attaching an arginine moiety to cellulose might be to react Na-Boc-Nw-(4-methoxy-2,3,6-trimethylbenzenesulfonyl)-L-(and/or D)-arginine this is also known as Na-Boc-Nw-MTR-L-(and/or D)-arginine with oxoyl chloride in an inert solvent such a methylene chloride to produce the acid chloride of Na-Boc-Nw-MTR-L-(and/or D)-arginine. The next step would be to stir and warm the acid chloride with cellulose, protected from air and water, either under nitrogen or with a drying tube. The next step would be to react the resulting compound(s) with tetrahydrofluoric acid in methylene chloride to produce cellulose with an attached arginine group. This way could also be used to attach other amino acid moieties (such as a lysine moiety and a cysteine moiety) to cellulose.

A cellulose polymer or a chitin polymer or a dendrimer polymer (or other nonsoluble carrier polymer—large enough that they will not go from dialysis machine to patient by going through the filtration membrane or by dissolving and entering the blood stream of the patient—or a non-biological oligomer) could be used and/or modified for use as a carrier molecule for 1) methylglyoxal, glyoxal, malondialdehyde, or similar molecules for use in hemodialysis of uremic patients to remove compounds with certain amino groupings such as arginine, lysine, cysteine or urea not to exclude other aminated compounds or 2) arginine or similar molecules for use in hemodialysis of diabetic patients to remove toxic dicarbonyl compounds such as methylglyoxal, glyoxal, deoxyglucasone and certain advanced glycation end-products.

Arginine could be attached to this polymer (a cellulose polymer or a chitin polymer or a dendrimer polymer (or other nonsoluble carrier polymer—large enough that they will not go from dialysis machine to patient by going through the filtration membrane or by dissolving and entering the blood stream of the patient—or a non-biological oligomer)) and the resulting compound used in a renal dialysis machine to remove toxic carbonyl and dicarbonyl compounds from the blood of diabetic patients on renal dialysis. Conversely, a dicarbonyl compound such as methylglyoxal could be attached to the same polymer (a cellulose polymer or a chitin polymer or a dendrimer polymer (or other nonsoluble carrier polymer—large enough that they will not go from dialysis machine to patient by going through the filtration membrane or by dissolving and entering the blood stream of the patient—or a non-biological oligomer)) and employed in dialysis machines to remove arginine, lysine, cysteine and nitrogenous waste products from the blood of uremic, liver failure patients.

The present invention includes a method of treating diabetic dialysis patients with a cellulose polymer or a chitin polymer or a dendrimer polymer (or other nonsoluble carrier polymer—large enough that they will not go from dialysis machine to patient by going through the filtration membrane or by dissolving and entering the blood stream of the patient—or a non-biological oligomer) modified with an arginine side chain, cysteine side chain and/or lysine side chain for removal of toxic carbonyl and/or dicarbonyl compounds such as methylglyoxal, glyoxal, malondialdehyde, deoxyglucasone and advanced glycation end-products by placement of the modified cellulose polymer or chitin polymer or a dendrimer polymer (or other nonsoluble carrier polymer—large enough that they will not go from dialysis machine to patient by going through the filtration membrane or by dissolving and entering the blood stream of the patient—or a non-biological oligomer) in the filtration system of the dialysis machine.

The present invention also includes a method of treating uremic dialysis patients with a cellulose polymer or a chitin polymer or a dendrimer polymer (or other nonsoluble carrier polymer—large enough that they will not go from dialysis machine to patient by going through the filtration membrane or by dissolving and entering the blood stream of the patient—or a non-biological oligomer) modified with a side chain consisting of a carbonyl, and/or dicarbonyl such as methylglyoxal, glyoxal, malondialdehyde, deoxyglucasone or phenylglyoxal for removal of aminated compounds such as arginine, cysteine, lysine, creatinine or urea not to exclude other aminated compounds by placement of the modified cellulose polymer or chitin polymer or dendrimer polymer (or other nonsoluble carrier polymer—large enough that they will not go from dialysis machine to patient by going through the filtration membrane or by dissolving and entering the blood stream of the patient—or a non-biological oligomer) in the filtration system of the dialysis machine.

The present invention includes a dialysis machine for use in treating diabetic dialysis patients, the dialysis machine having a filtration system, the filtration system having therein a cellulose polymer or a chitin polymer or a dendrimer polymer (or other nonsoluble carrier polymer—large enough that they will not go from dialysis machine to patient by going through the filtration membrane or by dissolving and entering the blood stream of the patient—or a non-biological oligomer) modified with an arginine side chain, cysteine side chain and/or lysine side chain for removal of toxic carbonyl and/or dicarbonyl compounds such as methylglyoxal, glyoxal, malondialdehyde, deoxyglucasone and advanced glycation end-products.

The present invention includes a dialysis machine for use in treating uremic dialysis patients, the dialysis machine having a filtration system, the filtration system having therein a cellulose polymer or a chitin polymer or a dendrimer polymer (or other nonsoluble carrier polymer—large enough that they will not go from dialysis machine to patient by going through the filtration membrane or by dissolving and entering the blood stream of the patient—or a non-biological oligomer) modified with a side chain consisting of a carbonyl, and/or dicarbonyl such as methylglyoxal, glyoxal, malondialdehyde, deoxyglucasone or phenylglyoxal for removal of aminated compounds such as arginine, cysteine, lysine, creatinine or urea not to exclude other aminated compounds.

One of ordinary skill in the art should be able to determine with which commercially available dialysis machines the filter of the present invention can be used.

All measurements disclosed herein are at standard temperature and pressure, at sea level on Earth, unless indicated otherwise. All materials used or intended to be used in a human being are biocompatible, unless indicated otherwise.

The foregoing embodiments are presented by way of example only; the scope of the present invention is to be limited only by the following claims.

1. A substance for use in a filtration system in a dialysis machine for use in treating diabetic dialysis patients, the substance comprising a phenylacetylene oligomer or other oligomer or macromolecule or dendrimer modified with an arginine side chain, cysteine side chain and/or lysine side chain for removal of toxic carbonyl and/or dicarbonyl compounds such as methylglyoxal, glyoxal, malondialdehyde, deoxyglucasone and advanced glycation end-products.

2. A substance for use in a filtration system in a dialysis machine for use in treating uremic dialysis patients, the substance comprising a phenylacetylene oligomer or other oligomer or macromolecule or dendrimer modified with a side chain consisting of a carbonyl, and/or dicarbonyl such as methylglyoxal, glyoxal, malondialdehyde, deoxyglucasone or phenylglyoxal for removal of aminated compounds such as arginine, cysteine, lysine, creatinine or urea not to exclude other aminated compounds.

3. A substance for use in a filtration system in a dialysis machine for use in treating diabetic dialysis patients, the substance comprising a cellulose polymer or a chitin polymer or a dendrimer polymer (or other nonsoluble carrier polymer—large enough that they will not go from dialysis machine to patient by going through the filtration membrane or by dissolving and entering the blood stream of the patient—or a non-biological oligomer) modified with an arginine side chain, cysteine side chain and/or lysine side chain for removal of toxic carbonyl and/or dicarbonyl compounds such as methylglyoxal, glyoxal, malondialdehyde, deoxyglucasone and advanced glycation end-products.

4. A substance for use in a filtration system in a dialysis machine for use in treating uremic dialysis patients, the substance comprising a cellulose polymer or a chitin polymer or a dendrimer polymer (or other nonsoluble carrier polymer—large enough that they will not go from dialysis machine to patient by going through the filtration membrane or by dissolving and entering the blood stream of the patient—or a non-biological oligomer) modified with a side chain consisting of a carbonyl, and/or dicarbonyl such as methylglyoxal, glyoxal, malondialdehyde, deoxyglucasone or phenylglyoxal for removal of aminated compounds such as arginine, cysteine, lysine, creatinine or urea not to exclude other aminated compounds.

5. The substance of claim 1, consisting essentially of the modified polymer, modified oligomer, modified macromolecule, or modified dendrimer.

6. The substance of claim 1, consisting of the modified polymer, modified oligomer, modified macromolecule, or modified dendrimer.

7. A filter for a dialysis machine using the substance of claim 1.

8. A filter for a dialysis machine consisting essentially of the substance of any one of claim 1.

9. A filter for a dialysis machine consisting of the substance of claim 1.

10. A dialysis machine using the filter of anyone of claim 7.

11. A method of treating a patient in need of treatment using the dialysis machine of claim 10.

12. A dialysis machine for use in treating diabetic dialysis patients, the dialysis machine having a filtration system, the filtration system having therein the substance of claim 1.

13. A dialysis machine for use in treating uremic dialysis patients, the dialysis machine having a filtration system, the filtration system having therein the substance of claim 2.

14. A dialysis machine for use in treating diabetic dialysis patients, the dialysis machine having a filtration system, the filtration system having therein the substance of claim 3.

15. A dialysis machine for use in treating uremic dialysis patients, the dialysis machine having a filtration system, the filtration system having therein the substance of claim 4.

16. A method of treating diabetic dialysis patients with the substance of claim 1 by placing the substance of claim 1 in filtration system of a dialysis machine by placement of the modified oligomer, macromolecule or dendrimer in the filtration system of the dialysis machine.

17. A method of treating uremic dialysis patients with the substance of claim 2 by placing the substance of claim 2 in a filtration system of a dialysis machine.

18. A method of treating diabetic dialysis patients with the substance of claim 3 by placing the substance of claim 3 in a filtration system of a dialysis machine.

19. A method of treating uremic dialysis patients with the substance of claim 4 by placing the substance of claim 4 in a filtration system of a dialysis machine.

20. (canceled)
